(19) United States
(12) Patent Application Publication
(10) Pub. No.: US 2023/0333105 A1
(43) Pub. Date: Oct. 19, 2023

(54) HIGH-THROUGHPUT FLOW CYTOMETRY ANALYSIS OF HIGHLY MULTIPLEXED SAMPLES USING SAMPLE INDEXING WITH SPECIFIC BINDING MEMBER-FLUOR CONJUGATES

(71) Applicant: Becton, Dickinson and Company, Franklin Lakes, NJ (US)

(72) Inventors: Jody Martin, Encinitas, CA (US); Adam Thomas Wright, La Jolla, CA (US); Ping He, San Diego, CA (US)

(21) Appl. No.: 18/133,908

(22) Filed: Apr. 12, 2023

Related U.S. Application Data
(60) Provisional application No. 63/332,085, filed on Apr. 18, 2022.

(51) Int. Cl. G01N 33/569; G01N 15/14; G01N 33/533

(52) U.S. Cl. CPC G01N 33/56966; G01N 15/1434; G01N 33/533; G01N 2015/1006

(57) ABSTRACT

Methods of producing a plurality of distinguishably fluorescently barcoded particle, e.g., cellular, bead, etc., samples, e.g., for use in the multiplex flow cytometric workflows, are provided. Aspects of the methods include: providing a plurality of particle, e.g., cellular, bead, etc., samples; and labeling different particle, e.g., cellular, bead, etc., samples of the plurality with unique fluorescent barcodes, wherein a given fluorescent barcode comprises one or more fluorescently labeled specific binding members that specifically bind to a particle marker. Also provided are compositions for practicing methods of the invention.

### CROSS-REFERENCE TO RELATED APPLICATIONS

Pursuant to 35 U.S.C. § 119 (e), this application claims priority to the filing dates of U.S. Provisional patent application Ser. No. 63/332,085 filed Apr. 18, 2022, the disclosure of which application is incorporated herein by reference in their entirety.

### INTRODUCTION

The characterization of analytes in biological fluids has become an important part of biological research, medical diagnoses and assessments of overall health and wellness of a patient. Detecting analytes in biological fluids, such as human blood or blood derived products, can provide results that may play a role in determining a treatment protocol of a patient having a variety of disease conditions.

Flow cytometry is a technique used to characterize and often times sort biological material, such as cells of a blood sample or particles of interest in another type of biological or chemical sample. A flow cytometer typically includes a sample reservoir for receiving a fluid sample, such as a blood sample, and a sheath reservoir containing a sheath fluid. The flow cytometer transports the particles (including cells) in the fluid sample as a cell stream to a flow cell, while also directing the sheath fluid to the flow cell. To characterize the components of the flow stream, the flow stream is irradiated with light. Variations in the materials in the flow stream, such as morphologies or the presence of fluorescent labels, may cause variations in the observed light and these variations allow for characterization and separation. To characterize the components in the flow stream, light must impinge on the flow stream and be collected. Light sources in flow cytometers can vary and may include one or more broad spectrum lamps, light emitting diodes as well as single wavelength lasers. The light source is aligned with the flow stream and an optical response from the illuminated particles is collected and quantified.

With high throughput flow cytometer workflows, there is a desire to process large numbers of different samples while minimizing reagent consumption and yet maximizing the robustness of the data obtained. Fluorescent Cell Barcoding (FCB) (Krutzik and Nolan, “Fluorescent cell barcoding in flow cytometry allows high-throughput drug screening and signaling profiling,” Nat. Methods (2006) 3(5):361-368) is a technique that was designed to meet this desire. In FCB, different cell samples are encoded with unique fluorescent signatures, following which the samples are combined or pooled for subsequent simultaneous antibody staining and data acquisition. In FCB, to encode different cell samples with unique fluorescent signatures, fluorescent dyes are derivatized to make them reactive with appropriate cellular targets, e.g., with N-hydroxysuccinimide so that they are reactive to amine functional groups present, e.g., on proteins, e.g., lysine side chains and at the N-terminus of proteins. Cell samples are stained with different concentrations of reactive fluorescent dye, which produces samples having unique dye intensity distributions. The resultant fluorescently encoded samples are distinguishable based on their fluorescence intensity in a barcoding detection channel. FCB eliminates sample-to-sample variations, which variations may arise from a number of a different sources, including staining volume, antibody concentration, etc. Furthermore, as many different samples are pooled for subsequent flow cytometric analysis, data acquisition times are minimized.

While FCB provides a number of benefits in flow cytometric workflows, such as mentioned above, it is not without disadvantages. FCB generally includes only 2 or 3 colors, and relies on complex reactive chemistries associated with dyes to chemically produce a covalent linkage of dyes at the cell membrane or within the cell. The materials are difficult to manufacture, store and ship, as they are highly unstable. The utility is fraught with complications due to inconsistency in labeling of cell samples. Resolution of more than 20 populations based on the fluorescent barcode is difficult.

### SUMMARY

The invention described here improves the manufacturability, stability, utility and multiplexibility of fluorescent cell barcoding, doing so by using well-characterized specific binding member, e.g., antibody, fluorophore conjugates. Embodiments of the invention provide a route to ultra-high throughput flow cytometry via fluorescence barcoding (sample indexing) followed by pooled sample data acquisition. The fluorescence barcoding is achieved with fluorophore conjugated specific binding members, e.g., antibodies, targeting a variety of ubiquitous cell surface epitopes. The ubiquitous nature of the cell surface features may be common to all cells, species specific, or targeted for certain subpopulations of cells of interest. With every sample in a particular study assigned a unique fluorescence signature, and/or brightness to each color in that signature, samples can be pooled and run concurrently as a single sample in multi-parameter flow cytometry. Embodiments described herein allow for 10s or 100s of samples to be pooled, assayed, and the multiplexed sample data acquired in a fraction of the time that it would take to acquire data individually from unpooled samples. Additionally, because each cell within a sample is indexed with the unique sample barcode, the concern about sample to sample contamination within the flow cytometer is mitigated. Individual sample data, based on specific fluorescence barcodes read from every cell associated with a given sample, may be bioinformatically identified and separated out from the single data file during post-acquisition analysis. The ability to combine 100s of samples into a single pool prior to assay and analysis as provided by embodiments of the invention has several additional advantages beyond much improved assay and analysis throughput. With single tube assay treatments and staining, tube to tube variation can be eliminated. The ability to load a single large multiplexed sample provides easier walk-away sample data collection automation compared to mechanical auto-loaders. Loading a single sample for all study data collection also minimizes human error during data collection across several tubes or wells.

While the above discussion is focused on cell barcoding, the invention is not so limited. Instead, the invention can be used to also barcode other types of particles, e.g., beads, enabling multiplexing of bead based assay samples.

Methods of producing a plurality of distinguishably fluorescently barcoded particle, e.g., cellular samples, e.g., for use in the multiplex flow cytometric workflows, are provided. Aspects of the methods include: providing a plurality of particle, e.g., cellular, samples; and labeling different particle, e.g., cellular, samples of the plurality with unique fluorescent barcodes, wherein a given fluorescent barcode comprises one or more fluorescently labeled specific binding members that specifically bind to a universal surface, e.g., cell surface, marker. Also provided are compositions for practicing methods of the invention.

### BRIEF DESCRIPTION OF THE FIGURES

The invention may be best understood from the following detailed description when read in conjunction with the accompanying drawing.

FIG. 1 provides a schematic illustration of a workflow according to an embodiment of the invention.

### DEFINITIONS

Unless defined otherwise, technical and scientific terms used herein have the same meaning as commonly understood by one of ordinary skill in the art to which the present disclosure belongs. See, e.g., Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, an antibody can be a full-length (e.g., naturally occurring or formed by normal immunoglobulin gene fragment recombinatorial processes) immunoglobulin molecule (e.g., an IgG antibody) or an immunologically active (i.e., specifically binding) portion of an immunoglobulin molecule, like an antibody fragment. In some embodiments, an antibody is a functional antibody fragment. For example, an antibody fragment can be a portion of an antibody such as F(ab′)2, Fab′, Fab, Fv, sFv and the like. An antibody fragment can bind with the same antigen that is recognized by the full-length antibody. An antibody fragment can include isolated fragments consisting of the variable regions of antibodies, such as the “Fv” fragments consisting of the variable regions of the heavy and light chains and recombinant single chain polypeptide molecules in which light and heavy variable regions are connected by a peptide linker (“scFv proteins”). Exemplary antibodies can include, but are not limited to, antibodies for cancer cells, antibodies for viruses, antibodies that bind to cell surface receptors (for example, CD8, CD34, and CD45), and therapeutic antibodies.

As used herein the term “associated” or “associated with” can mean that two or more species are identifiable as being co-located at a point in time. An association can mean that two or more species are or were within a similar container. An association can be an informatics association. For example, digital information regarding two or more species can be stored and can be used to determine that one or more of the species were co-located at a point in time. An association can also be a physical association. In some embodiments, two or more associated species are “tethered”, “attached”, or “immobilized” to one another or to a common solid or semisolid surface. An association may refer to covalent or non-covalent means for attaching labels to solid or semi-solid supports such as beads. An association may be a covalent bond between a target and a label. An association can comprise hybridization between two molecules (such as a target molecule and a label).

As used herein, the term “complementary” can refer to the capacity for precise pairing between two nucleotides. For example, if a nucleotide at a given position of a nucleic acid is capable of hydrogen bonding with a nucleotide of another nucleic acid, then the two nucleic acids are considered to be complementary to one another at that position. Complementarity between two single-stranded nucleic acid molecules may be “partial,” in which only some of the nucleotides bind, or it may be complete when total complementarity exists between the single-stranded molecules. A first nucleotide sequence can be said to be the “complement” of a second sequence if the first nucleotide sequence is complementary to the second nucleotide sequence. A first nucleotide sequence can be said to be the “reverse complement” of a second sequence, if the first nucleotide sequence is complementary to a sequence that is the reverse (i.e., the order of the nucleotides is reversed) of the second sequence. As used herein, the terms “complement”, “complementary”, and “reverse complement” can be used interchangeably. It is understood from the disclosure that if a molecule can hybridize to another molecule it may be the complement of the molecule that is hybridizing.

As used herein, the term “sample” can refer to a composition comprising targets. Suitable samples for analysis by the disclosed methods, devices, and systems include cells, tissues, organs, or organisms. A cellular sample is a composition that is made up of multiple cells, such as a composition that includes multiple disparate cells, such as an aqueous composition of single cells, where the number of cells may vary.

As used herein, the term “sampling device” or “device” can refer to a device which may take a section of a sample and/or place the section on a substrate. A sample device can refer to, for example, a fluorescence activated cell sorting (FACS) machine, a cell sorter machine, a biopsy needle, a biopsy device, a tissue sectioning device, a microfluidic device, a blade grid, and/or a microtome.

As used herein, the term “solid support” can refer to discrete solid or semi-solid surfaces to which nucleic acids may be attached. A solid support may encompass any type of solid, porous, or hollow sphere, ball, bearing, cylinder, or other similar configuration composed of plastic, ceramic, metal, or polymeric material (e.g., hydrogel) onto which a nucleic acid may be immobilized (e.g., covalently or non-covalently). A solid support may comprise a discrete particle that may be spherical (e.g., microspheres) or have a non-spherical or irregular shape, such as cubic, cuboid, pyramidal, cylindrical, conical, oblong, or disc-shaped, and the like. A bead can be non-spherical in shape. A plurality of solid supports spaced in an array may not comprise a substrate. A solid support may be used interchangeably with the term “bead.” A bead sample is a composition that is made up of multiple different beads, which beads may be distinguishable from each based on size and/or fluorescent signature (e.g., as provided by emission maximum and/or brightness), where different beads may specific bind to different analytes, e.g., proteins, where the number of beads may vary.

### DETAILED DESCRIPTION

Methods of producing a plurality of distinguishably fluorescently barcoded particle, e.g., cellular, samples, e.g., for use in the multiplex flow cytometric workflows, are provided. Aspects of the methods include: providing a plurality of particle, e.g., cellular, samples; and labeling different particle, e.g., cellular, samples of the plurality with unique fluorescent barcodes, wherein a given fluorescent barcode comprises one or more fluorescently labeled specific binding members that specifically bind to a universal particle surface, e.g., cell surface, marker. Also provided are compositions for practicing methods of the invention.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the system and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods

As summarized above, methods of producing a plurality of distinguishably fluorescently barcoded particle, e.g., cellular or bead, samples are provided. By “fluorescently barcoded sample” is meant a particle, e.g., cellular, bead, etc., sample made up of a plurality of particles, e.g., cells, beads, etc., where the particles, e.g., cells, beads, etc., of the sample are associated the same fluorescent barcode. Because the particles, e.g., cells, beads, etc., of the sample are associated with the same fluorescent barcode, they comprise a common fluorescent barcode, which barcode can be detected during flow cytometric analysis and used to determine from which particle, e.g., cellular, bead, etc., sample a given particle, e.g., cell, bead, etc., cell originated. The fluorescent barcode of a given fluorescently barcoded sample can be used to obtain a fluorescent signature (i.e., fluorescent identifier) made up of one or more fluorescent emission signals obtained from one or more fluorophores of the fluorescent barcode associated with particles, e.g., cells, beads, etc., of the sample, e.g., as described in greater detail below. Different fluorescently barcoded samples of the plurality produced by methods of embodiments of the invention have distinguishable fluorescent barcodes associated therewith, and therefore provide different fluorescent signatures, e.g., when assayed by flow cytometric protocols.

As indicated above, a fluorescent barcode of the invention comprises one or more one or more fluorescently labeled specific binding members that specifically bind to particle surface marker, e.g., a universal cell marker, a marker present on a surface of a bead, etc. Where a given fluorescent barcode includes more than one fluorescently labeled specific binding members, the two or more fluorescently labeled specific binding members collectively make up the fluorescent barcode. As such, a given fluorescent barcode may, in embodiments of the invention, be made up of a single fluorescently labeled specific binding member, or two or more fluorescently labeled specific binding members, e.g., 2 to 20, such as 3 to 10, fluorescently labeled specific binding members, which collectively make up the fluorescent barcode. Any given two distinguishable fluorescent barcodes may be distinguishable from each other (and give rise to distinguishable fluorescent signatures) based on the types of fluorophores and/or signal brightness provided thereby. As such, any two distinguishable fluorescent signatures obtained from different barcodes may be distinguishable based on fluorescent signals and/or intensity thereof, of the fluorescent signals collectively making up the fluorescent signature. For example, two distinguishable fluorescent barcodes may be distinguishable from each other because they are made up of combinations of different types fluorophores, e.g., where one includes fluorophores a, b and c and the other includes fluorophores b, c and d. Two distinguishable fluorescent barcodes may also be distinguishable from each other because they are made up of different amounts of fluorophores, e.g., where one is made up of fluorophores a, b and c present in a first amount on the various specific binding members and the other is made up of fluorophores present at a second amount that differs from the first amount at a value that can be detected, e.g., by a difference in brightness of signal. Combinations of type and amount of fluorophores may be employed to provide any desired number of unique fluorescent barcodes. As summarized above, methods of embodiments of the invention provide for a plurality of distinguishably fluorescently barcoded particle, e.g., cellular, bead, etc., samples. While the number of a distinguishably fluorescent barcoded particle, e.g., cellular, bead, etc., samples produced in a given embodiment may vary, in some instances the number ranges from 5 to 5000, such as 5 to 500, including 50 to 400 particle, e.g., cellular, bead, etc., samples, where in some instances number of particle, e.g., cellular, bead, etc., samples corresponds to the number of wells of a conventional multi-well plate, such as 6, 12, 24, 48, 96 or 384 particle, e.g., cellular, bead, etc., samples.

In practicing embodiments of the methods, a plurality of particle, e.g., cellular, bead, etc., samples to be fluorescently barcoded is provided. As reviewed above, while the number of particle, e.g., cellular, bead, etc., samples may vary, in some instances the number ranges from 5 to 5000, such as 5 to 500, including 50 to 400 particle, e.g., cellular, bead, etc., samples, where in some instances number of particle, e.g., cellular, bead, etc., samples corresponds to the number of wells of a conventional multi-well plate, such as 6, 12, 24, 48, 96 or 384 particle, e.g., cellular, bead, etc., samples. The number of particles, e.g., cells, beads, etc., in a given particle, e.g., cellular, bead, etc., sample may vary, wherein in some instances the number of particles, e.g., cells, beads, etc., ranges from 50 to 50,000,000, such as 100 to 1,000,000 and including 500 to 100,000.

As summarized above, particles in a given particle sample may vary, where examples of particles include cells, beads, etc. Cells present in a given cellular sample may be any type of cell, including prokaryotic and eukaryotic cells. Suitable prokaryotic cells include, but are not limited to, bacteria such as E. coli, various Bacillus species, and the extremophile bacteria such as thermophiles, etc. Suitable eukaryotic cells include, but are not limited to, fungi such as yeast and filamentous fungi, including species of Aspergillus, Trichoderma, and Neurospora; plant cells including those of corn, sorghum, tobacco, canola, soybean, cotton, tomato, potato, alfalfa, sunflower, etc.; and animal cells, including fish, birds and mammals. Suitable fish cells include, but are not limited to, those from species of salmon, trout, tilapia, tuna, carp, flounder, halibut, swordfish, cod and zebrafish. Suitable bird cells include, but are not limited to, those of chickens, ducks, quail, pheasants and turkeys, and other jungle foul or game birds. Suitable mammalian cells include, but are not limited to, cells from horses, cows, buffalo, deer, sheep, rabbits, rodents such as mice, rats, hamsters and guinea pigs, goats, pigs, primates, marine mammals including dolphins and whales, as well as cell lines, such as human cell lines of any tissue or stem cell type, and stem cells, including pluripotent and non-pluripotent, and non-human zygotes.

Suitable cells also include those cell types implicated in a wide variety of disease conditions, even while in a non-diseased state. Accordingly, suitable eukaryotic cell types include, but are not limited to, tumor cells of all types (e.g., melanoma, myeloid leukemia, carcinomas of the lung, breast, ovaries, colon, kidney, prostate, pancreas and testes), cardiomyocytes, dendritic cells, endothelial cells, epithelial cells, lymphocytes (T-cell and B cell), mast cells, eosinophils, vascular intimal cells, macrophages, natural killer cells, erythrocytes, hepatocytes, leukocytes including mononuclear leukocytes, stem cells such as haemopoietic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. In certain embodiments, the cells are primary disease state cells, such as primary tumor cells. Suitable cells also include known research cells, including, but not limited to, Jurkat T cells, NIH3T3 cells, CHO, COS, etc. See the ATCC cell line catalog, hereby expressly incorporated by reference.

In certain embodiments, the cells used in the present invention are taken from a subject. As used herein “subject” refers to both human and other animals as well as other organisms, such as experimental animals. Thus, the methods and compositions described herein are applicable to both human and veterinary applications. In certain embodiments the subject is a mammal, including embodiments in which the subject is a human patient either having (or suspected of having) a disease or pathological condition.

In certain embodiments, the cells being analyzed are enriched prior to fluorescent barcoding, e.g., as described in greater detail below. For example, if the cells of interest are white blood cells derived from a human subject, whole blood from the subject may be subjected to density gradient centrifugation to enrich for peripheral blood mononuclear cells (PBMCs, or white blood cells). Cells may be enriched using any convenient method known in the art, including fluorescence activated cell sorting (FACS), magnetically activated cell sorting (MACS), density gradient centrifugation and the like. Parameters employed for enriching certain cells from a mixed population include, but are not limited to, physical parameters (e.g., size, shape, density, etc.), in vitro growth characteristics (e.g., in response to specific nutrients in cell culture), and molecule expression (e.g., expression of cell surface proteins or carbohydrates, reporter molecules, e.g., green fluorescent protein, etc.).

In certain embodiments, the cells are live cells which retain viability during the course of the assay. By “retain viability” is meant that a certain percentage of the cells remain alive at the conclusion of the assay, including from about 20% viable up to and including about 100% viable. In certain other embodiments, the methods of the present invention are carried out in such a manner as the cells are rendered non-viable during the course of the assay, e.g., the cells may be fixed, permeabilized, or otherwise maintained in buffers or under conditions in which the cells do not survive. Such parameters are generally dictated by the nature of the assay being performed as well as the reagents being employed.

In some instances the cells may be treated, e.g., with a stimulus. Stimuli with which cells may be treated may vary, ranging from culture conditions, exposure to changes in temperature, e.g., heat or cold, exposure to electromagnetic radiation, e.g., light, exposure to active agents, exposure to mechanical changes, etc. As desired, different cellular samples of the plurality may be treated with the same or different stimulus. As such, in some instances the method includes differentially treating two or more of the plurality of cellular samples, e.g., where two or more different sample are contacted with different active agents, or different concentrations of the same active agent, etc.

Particle samples employed in embodiments of the invention may be bead samples. Bead samples may include one or more distinguishable beads, where each of the one or more distinguishable beads specifically binds to a different analyte, e.g., protein, nucleic acid, small molecule, etc. The number of distinguishable beads in a given bead sample may vary, where in some instances the number ranges from 1 to 250, such as 1 to 100, such as 1 to 50, e.g., 1 to 30. In some embodiments, bead samples are samples prepared by combination of a biological sample, e.g., blood-based sample, such as plasma sample, with one or more beads that specifically bind to an analyte of interest, where in some instances the beads may be beads of a multiplex bead array assay, such as beads of Cytometric Bead Array (CBA) (e.g., as commercialized by BD Biosciences), beads of Luminex xMAP (ThermoFisher), etc. Multiplex bead array assays are and bead systems usable therein, which may be barcoded and processed in accordance with embodiments of the invention, include those further reviewed in Elshal & McCoy, “Multiplex Bead Array Assays: Performance Evaluation and Comparison of Sensitivity to ELISA,” Methods. 2006 April; 38(4): 317-323 PMID: 16481199; and Zhang et al., “Cytometry Multiplex Bead Antibody Array,” Methods Mol Biol. 2021; 2237:83-92; PMID 33237410.

The plurality of particle, e.g., cellular, bead, etc., samples may be present in individual particle, e.g., cellular, bead, etc., composition containers. Particle, e.g., cellular, bead, etc., composition containers may be configured to hold aqueous particle, e.g., cellular, bead, etc., compositions, and may have any convenient volume, where the volume may range in some instances from 10 μl to 5 ml, such as 10 μl to 1 ml. Particle, e.g., cellular, bead, etc., containers of interest that may be employed to hold particle, e.g., cellular, bead, etc., compositions may vary, and include tubes, vials, wells, e.g., of multi-well plates, etc., where in the some instances the particle, e.g., cellular, bead, etc., compositions are present in wells of a standard laboratory multi-well plate, e.g., a 96- or 384-well plate.

The plurality of samples may be provided using any convenient protocol. In some instances, an initial particle, e.g., cellular, bead, etc., sample may be divided into the plurality of particle, e.g., cellular, bead, etc., samples. In yet other instances, one or more of the samples of the plurality, including all members of the plurality may be obtained from different sources. In yet other embodiments, subsets of the particle, e.g., cellular, bead, etc., samples may be prepared from the same initial source.

Following provision of the plurality of particle, e.g., cellular, bead, etc., samples, particle, e.g., cellular, bead, etc., samples of the plurality are fluorescently barcoded. To fluorescently barcode particle, e.g., cellular, bead, etc., samples of the plurality, embodiments of the methods include labeling each of the particle, e.g., cellular, bead, etc., samples of the plurality to be barcoded with a unique fluorescent barcode. A uniquely fluorescently barcoded sample is a sample of the plurality that has a fluorescent barcode that is different from any other fluorescent barcode of any other sample of the plurality. As such, a given fluorescent barcode of one labeled particle, e.g., cellular, bead, etc., sample of the plurality is distinguishable from the fluorescent barcodes of any other particle, e.g., cellular, bead, etc., sample of the plurality.

As summarized above, a given fluorescent barcode includes one or more fluorescently labeled specific binding members that specifically bind to a particle marker, e.g., universal cell marker, a surface marker on a bead, etc. In some instances, a given fluorescent barcode includes a single fluorescently labeled specific binding member that specifically binds to a marker, e.g., universal cell marker, a marker on a surface of a bead. In yet other instances, a given fluorescent barcode includes a plurality of distinguishably fluorescently labeled specific binding members that each bind to different marker, e.g., different universal cell markers, different markers on a surface of a bead, etc. In such instances, the number of different distinguishably fluorescently labeled specific binding members making up a given barcode may vary, ranging in some instances from 2 to 20 distinguishably fluorescently labeled specific binding members, such as from 3 to 10 distinguishably fluorescently labeled specific binding members.

Where a given fluorescent barcode is made up of two or more distinguishably fluorescently labeled specific binding members, the distinguishably fluorescently labeled specific binding members differ from each other by emission maximum, e.g., as provided by different types of fluorophores on different specific binding members. As such, any two of the labeled specific binding members may differ from each other by emission maximum, e.g., as provided by different fluorophores. The plurality of two or more distinguishably fluorescently labeled specific binding members in such instances collectively makes up the fluorescent barcode for the sample.

Distinguishably fluorescently labeled specific binding members that make up fluorescent barcodes of the invention include a specific binding member and a fluorescent label. The specific binding member components of the fluorescently labeled specific binding members that make up fluorescent barcodes employed in embodiments of the invention may vary. The term “specific binding” refers to a direct association between two molecules, due to, for example, covalent, electrostatic, hydrophobic, and ionic and/or hydrogen-bond interactions, including interactions such as salt bridges and water bridges. A specific binding member describes a member of a pair of molecules which have binding specificity for one another. The members of a specific binding pair may be naturally derived or wholly or partially synthetically produced. One member of the pair of molecules has an area on its surface, or a cavity, which specifically binds to and is therefore complementary to a particular spatial and polar organization of the other member of the pair of molecules. Thus, the members of the pair have the property of binding specifically to each other. Examples of pairs of specific binding members are antigen-antibody, biotin-avidin, hormone-hormone receptor, receptor-ligand, enzyme-substrate. Specific binding members of a binding pair exhibit high affinity and binding specificity for binding with each other. Typically, affinity between the specific binding members of a pair is characterized by a K_{d} (dissociation constant) of 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, including 10⁻¹⁵ M or less. “Affinity” refers to the strength of binding, increased binding affinity being correlated with a lower KD. In an embodiment, affinity is determined by surface plasmon resonance (SPR), e.g., as used by Biacore systems. The affinity of one molecule for another molecule is determined by measuring the binding kinetics of the interaction, e.g., at 25° C. “Affinity” refers to the strength of binding, increased binding affinity being correlated with a lower KD. In an embodiment, affinity is determined by surface plasmon resonance (SPR), e.g., as used by Biacore systems. The affinity of one molecule for another molecule is determined by measuring the binding kinetics of the interaction, e.g., at 25° C. Specific binding members may vary, where examples of specific binding members include, but are not limited to, polypeptides, nucleic acids, carbohydrates, lipids, peptoids, etc. In some instances, the specific binding member is proteinaceous. As used herein, the term “proteinaceous” refers to a moiety that is composed of amino acid residues. A proteinaceous moiety can be a polypeptide. In certain cases, the proteinaceous specific binding member is an antibody. In certain embodiments, the proteinaceous specific binding member is an antibody fragment, e.g., a binding fragment of an antibody that specifically binds to a polymeric dye. As used herein, the terms “antibody” and “antibody molecule” are used interchangeably and refer to a protein consisting of one or more polypeptides substantially encoded by all or part of the recognized immunoglobulin genes. The recognized immunoglobulin genes, for example in humans, include the kappa (k), lambda (l), and heavy chain genetic loci, which together comprise the myriad variable region genes, and the constant region genes mu (u), delta (d), gamma (g), sigma (e), and alpha (a) which encode the IgM, IgD, IgG, IgE, and IgA isotypes respectively. An immunoglobulin light or heavy chain variable region consists of a “framework” region (FR) interrupted by three hypervariable regions, also called “complementarity determining regions” or “CDRs”. The extent of the framework region and CDRs have been precisely defined (see, “Sequences of Proteins of Immunological Interest,” E. Kabat et al., U.S. Department of Health and Human Services, (1991)). The numbering of all antibody amino acid sequences discussed herein conforms to the Kabat system. The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen. The term antibody is meant to include full length antibodies and may refer to a natural antibody from any organism, an engineered antibody, or an antibody generated recombinantly for experimental, therapeutic, or other purposes as further defined below. Antibody fragments of interest include, but are not limited to, Fab, Fab′, F(ab′)2, Fv, scFv, or other antigen-binding subsequences of antibodies, either produced by the modification of whole antibodies or those synthesized de novo using recombinant DNA technologies. Antibodies may be monoclonal or polyclonal and may have other specific activities on cells (e.g., antagonists, agonists, neutralizing, inhibitory, or stimulatory antibodies). It is understood that the antibodies may have additional conservative amino acid substitutions which have substantially no effect on antigen binding or other antibody functions. In certain embodiments, the specific binding member is a Fab fragment, a F(ab′)₂ fragment, a scFv, a diabody or a triabody. In certain embodiments, the specific binding member is an antibody. In some cases, the specific binding member is a murine antibody or binding fragment thereof. In certain instances, the specific binding member is a recombinant antibody or binding fragment thereof.

The specific binding members that make up the fluorescent barcodes may specifically bind to any convenient binding member pair, such as a particle marker, e.g., a protein on a surface of a particle, such as a cell or bead. Where the particle sample is a cellular sample, in some instances the specific binding members that make up the fluorescent barcodes specifically bind to universal cell markers. In some instances, the universal marker is a cell surface marker, where cell surface markers of interest include, but are not limited to, ubiquitous cell surface markers, i.e., cell surface markers that are at least predicted to be on all cells of a given cellular sample to be processed in a given workflow in accordance with the present invention. Examples of ubiquitous cell surface markers to which specific binding members may specifically bind include, but are not limited to: CD44, CD45, CD47, β-2 microglobulin, and the like. Where a given barcode is made up of two or more fluorescently labeled specific binding members, each of the two or more fluorescent labeled specific binding member may specifically bind to a different universal marker, as desired. As such, if a given fluorescent barcode is made up of 4 distinguishably labeled specific binding members, the distinguishably labeled specific binding members may bind to four different universal markers, e.g., one may be bind to CD44, one may bind to CD45, one may be bind to CD47 and one may be bind to β-2 micro-globulin.

Where the particle sample is a bead sample, in some instances the specific binding members that make up the fluorescent barcodes specifically bind to markers present on the surface of the beads. In some instances, the marker or markers present on the surface of a given bead are markers that are different from the analyte specific binding member of the bead, where examples of such markers include proteins or fragments thereof that are different from the specific binding members, e.g., antibodies, of the bead, and do not interfere in the desired function of the specific binding member being able to specifically bind to its target analyte. In some instances, the marker(s) may be a ubiquitous cell surface marker, e.g., as described above, such as CD44, CD45, CD47, β-2 microglobulin, and the like. Where a given barcode is made up of two or more fluorescently labeled specific binding members, each of the two or more fluorescent labeled specific binding member may specifically bind to a different bead marker, as desired. As such, if a given fluorescent barcode is made up of 4 distinguishably labeled specific binding members, the distinguishably labeled specific binding members may bind to four different bead markers, e.g., one may be bind to CD44, one may bind to CD45, one may be bind to CD47 and one may be bind to β-2 micro-globulin.

In addition to the specific binding member component, the fluorescently labeled specific binding members that make up fluorescent barcodes include fluorescent labels. A given fluorescent label may include one or more fluorophores, as desired. As such, a given specific binding member may be labeled with fluorescent label that includes a single type of fluorophore. Alternatively, a given specific binding member may be labeled with a fluorescent label that includes two or more different types fluorophores, e.g., as found in tandem dyes, e.g., where a first fluorophore acts as a donor to a second fluorophore. Examples of fluorophores include, but are not limited to: acridine and derivatives such as acridine, acridine orange, acridine yellow, acridine red, and acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-amino-1-naphthyl)maleimide; anthranilamide; Brilliant Yellow; coumarin and derivatives such as coumarin, 7-amino-4-methylcoumarin (AMC, Coumarin 120), 7-amino-4-trifluoromethylcouluarin (Coumaran 151); cyanine and derivatives such as cyanosine, Cy3, Cy5, Cy5.5, and Cy7; 4′,6-diamidino-2-phenylindole (DAPI); 5′,5″-dibromopyrogallol-sulfonephthalein (Bromopyrogallol Red); 7-diethylamino-3-(4′-isothiocyanatophenyl)-4-methylcoumaran; diethylaminocoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein isothiocyanate (FITC), fluorescein chiorotriazinyl, naphthofluorescein, and QFITC (XRITC); fluorescamine; IR144; IR1446; Lissamine™; Lissamine rhodamine, Lucifer yellow; Malachite Green isothiocyanate; 4-methylumbelliferone; ortho cresolphthalein; nitrotyrosine; pararosaniline; Nile Red; Oregon Green; Phenol Red; B-phycoerythrin; o-phthaldialdehyde; pyrene and derivatives such as pyrene, pyrene butyrate and succinimidyl 1-pyrene butyrate; Reactive Red 4 (Cibacron™ Brilliant Red 3B-A); rhodamine and derivatives such as 6-carboxy-X-rhodamine (ROX), 6-carboxyrhodamine (R6G), 4,7-dichlororhodamine lissamine, rhodamine B sulfonyl chloride, rhodamine (Rhod), rhodamine B, rhodamine 123, rhodamine X isothiocyanate, sulforhodamine B, sulforhodamine 101, sulfonyl chloride derivative of sulforhodamine 101 (Texas Red), N,N,N′,N′-tetramethyl-6-carboxyrhodamine (TAMRA), tetramethyl rhodamine, and tetramethyl rhodamine isothiocyanate (TRITC); riboflavin; rosolic acid and terbium chelate derivatives; xanthene; Alexa-Fluor dyes (e.g., Alexa Fluor 350, Alexa Fluor 430, Alexa Fluor 488, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, Alexa Fluor 750), Pacific Blue, Pacific Orange, Cascade Blue, Cascade Yellow; Quantum Dot dyes (Quantum Dot Corporation); Dylight dyes from Pierce (Rockford, IL), including Dylight 800, Dylight 680, Dylight 649, Dylight 633, Dylight 549, Dylight 488, Dylight 405; or combinations thereof. Other fluorophores or combinations thereof known to those skilled in the art may also be used, for example those available from Molecular Probes (Eugene, Oreg.) and Exciton (Dayton, Ohio).

In some instances, a specific binding member is labeled with one or more polymeric dyes (e.g., fluorescent polymeric dyes). Fluorescent polymeric dyes that find use in the subject methods and systems are varied. In some instances of the method, the polymeric dye includes a conjugated polymer. Conjugated polymers (CPs) are characterized by a delocalized electronic structure which includes a backbone of alternating unsaturated bonds (e.g., double and/or triple bonds) and saturated (e.g., single bonds) bonds, where π-electrons can move from one bond to the other. As such, the conjugated backbone may impart an extended linear structure on the polymeric dye, with limited bond angles between repeat units of the polymer. For example, proteins and nucleic acids, although also polymeric, in some cases do not form extended-rod structures but rather fold into higher-order three-dimensional shapes. In addition, CPs may form “rigid-rod” polymer backbones and experience a limited twist (e.g., torsion) angle between monomer repeat units along the polymer backbone chain. In some instances, the polymeric dye includes a CP that has a rigid rod structure. The structural characteristics of the polymeric dyes can have an effect on the fluorescence properties of the molecules.

Any convenient polymeric dye may be utilized in the subject devices and methods. In some instances, a polymeric dye is a multichromophore that has a structure capable of harvesting light to amplify the fluorescent output of a fluorophore. In some instances, the polymeric dye is capable of harvesting light and efficiently converting it to emitted light at a longer wavelength. In some cases, the polymeric dye has a light-harvesting multichromophore system that can efficiently transfer energy to nearby luminescent species (e.g., a “signaling chromophore”). Mechanisms for energy transfer include, for example, resonant energy transfer (e.g., Forster (or fluorescence) resonance energy transfer, FRET), quantum charge exchange (Dexter energy transfer), and the like. In some instances, these energy transfer mechanisms are relatively short range; that is, close proximity of the light harvesting multichromophore system to the signaling chromophore provides for efficient energy transfer. Under conditions for efficient energy transfer, amplification of the emission from the signaling chromophore occurs when the number of individual chromophores in the light harvesting multichromophore system is large; that is, the emission from the signaling chromophore is more intense when the incident light (the “excitation light”) is at a wavelength which is absorbed by the light harvesting multichromophore system than when the signaling chromophore is directly excited by the pump light.

The multichromophore may be a conjugated polymer. Conjugated polymers (CPs) are characterized by a delocalized electronic structure and can be used as highly responsive optical reporters for chemical and biological targets. Because the effective conjugation length is substantially shorter than the length of the polymer chain, the backbone contains a large number of conjugated segments in close proximity. Thus, conjugated polymers are efficient for light harvesting and enable optical amplification via Forster energy transfer.

Polymeric dyes of interest include, but are not limited to, those dyes described in U.S. Pat. Nos. 7,270,956; 7,629,448; 8,158,444; 8,227,187; 8,455,613; 8,575,303; 8,802,450; 8,969,509; 9,139,869; 9,371,559; 9,547,008; 10,094,838; 10,302,648; 10,458,989; 10,641,775 and 10,962,546 the disclosures of which are herein incorporated by reference in their entirety; and Gaylord et al., J. Am. Chem. Soc., 2001, 123 (26), pp 6417-6418; Feng et al., Chem. Soc. Rev., 2010,39, 2411-2419; and Traina et al., J. Am. Chem. Soc., 2011, 133 (32), pp 12600-12607, the disclosures of which are herein incorporated by reference in their entirety. Specific polymeric dyes that may be employed include, but are not limited to, BD Horizon Brilliant™ Dyes, such as BD Horizon Brilliant™ Violet Dyes (e.g., BV421, BV510, BV605, BV650, BV711, BV786); BD Horizon Brilliant™ Ultraviolet Dyes (e.g., BUV395, BUV496, BUV737, BUV805); and BD Horizon Brilliant™ Blue Dyes (e.g., BB515) (BD Biosciences, San Jose, CA). Any fluorochromes that are known to a skilled artisan-including, but not limited to, those described above—or are yet to be discovered may be employed in the subject methods.

In some instances, each of the plurality of distinguishably fluorescently labeled specific binding members that make up a given barcode is excitable by common light source, such as a common laser. In such instances, each of the plurality of distinguishably fluorescently labeled specific binding members that make up a given barcode may have a common excitation maximum, but differ from each other in terms of emission maximum.

As reviewed above, any given two distinguishable fluorescent barcodes may be distinguishable from each other (and give rise to distinguishable fluorescent signatures) based on the types of fluorophores making up the barcode and/or signal brightness provided thereby. As such, any two distinguishable fluorescent signatures obtained from different barcodes may be distinguishable based on fluorescent signals and/or intensity thereof, of the fluorescent signals collectively making up the fluorescent signature. For example, two distinguishable fluorescent barcodes may be distinguishable from each other because they are made up of combinations of different types fluorophores, e.g., where one includes fluorophores a, b and c and the other includes fluorophores b, c and d. Two distinguishable fluorescent barcodes may also be distinguishable from each other because they are made up of different amounts of fluorophores, e.g., where one is made up of fluorophores a, b and c present in a first amount on the various specific binding members and the other is made up of fluorophores present at a second amount that differs from the first amount at a value that can be detected, e.g., by a difference in brightness of signal. Different brightnesses may readily be provided by having differing amounts of fluorophores associated with the specific binding members. Combinations of type and amount of fluorophores may be employed to provide any desired number of unique fluorescent barcodes. As summarized above, methods of embodiments of the invention provide for a plurality of distinguishably fluorescently barcoded particle, e.g., cellular, bead, etc., samples.

A fluorescent barcode may be associated with a given particle, e.g., cellular, bead, etc., sample, such that the particle, e.g., cellular, bead, etc., sample is labeled with the fluorescent barcode, using any convenient protocol. For example, a particle, e.g., cellular, bead, etc., sample may be contacted with a barcode labeling composition that includes the different fluorescently labeled specific binding members that collectively make up the barcode for that composition. In other instances, the particle, e.g., cellular, bead, etc., sample may be sequentially contacted with the different fluorescently labeled specific binding members that make up the barcode for that sample. For example, to label a plurality of particle, e.g., cellular, bead, etc., samples, all samples that include a given specific labeled specific binding member in their intended fluorescent barcodes may be first contacted with that labeled specific binding member. Then, all samples that include a second a given specific labeled specific binding member in their intended fluorescent barcodes may be contacted with that labeled specific binding member, where one or more samples that are contacted with the second labeled specific binding member may be samples that were also contacted with the first labeled specific binding member, dependent on the fluorescent barcode for those samples. For example, in those samples having a fluorescent barcode that includes both the first and second binding members, those samples will be contacted with both the first and second labeled specific binding members. Contact may be achieved under any suitable conditions that provide for specific binding of the fluorescently labeled specific binding members to their correspondence universal markers. The labeled specific binding members may be contacted with particles, e.g., cells, bead, etc., of the cellular samples, e.g., by introducing the labeled specific binding members into the containers of the particle, e.g., cellular, bead, etc., samples, such as by manual or automated fluid dispensing. In some instances, an automated liquid dispensing system may be employed to dispense different fluorescently labeled binding members in different combinations into different particle, e.g., cellular, bead, etc., samples to provide for the distinguishably fluorescently barcoded samples.

Following production of the plurality of distinguishably fluorescently barcoded particle, e.g., cellular, bead, etc., samples, the resultant plurality may be pooled, as desired, for subsequent processing. As such, the disparate fluorescently barcoded samples may be combined into a single composition. A single composition may be prepared from the different fluorescently barcoded particle, e.g., cellular, bead, etc., samples using any convenient protocol, such as by transferring the contents of each container, e.g., well of a well-plate, to single container of suitable volume, e.g., tube or vial. The resultant pooled composition may then further processed, as desired.

In certain embodiments of the present invention, e.g., where the particle samples are cellular sample, the methods may include detection of one or more phenotype characteristics of the cells, which phenotype characteristics are separate from the fluorescent barcode. Detectable phenotypic characteristics include, but are not limited to, presence of an analyte, e.g., cell surface or internal marker, physical characteristic (e.g., size, shape, granularity, etc.), cell number (or frequency), etc. Virtually any detectable characteristic of interest can be assayed for as the detectable phenotypic characteristic of interest. In certain embodiments, the methods of the present invention are drawn to detecting the presence of an analyte, e.g., a marker, associated with (e.g., in, on, or attached to) the cells being assayed, either qualitatively or quantitatively.

In certain of these embodiments, the method includes contacting the combined or pooled cell sample with a detectable analyte-specific binding agent. By “analyte-specific binding agent” and grammatical equivalents thereof, is meant any molecule, e.g., nucleic acids, small organic molecules, and proteins, nucleic acid binding dye (e.g., ethidium bromide) which are capable of associating with a specific analyte (or specific isoform of an analyte) in a cell over any others. Analytes of interest include any molecule associated with or present within the cells being analyzed in the subject methods. As such, analytes of interest include, but are not limited to, proteins, carbohydrates, organelles, nucleic acids, infectious particles (e.g., viruses, bacteria, parasites), metabolites, etc. In certain embodiments, the analyte-specific binding agent is a protein. In certain of these embodiments, the analyte-specific binding agent is an antibody or binding fragment thereof, e.g., as described above. Accordingly, the methods and compositions of the present invention may be used to detect any particular element isoform in a sample that is antigenically detectable and antigenically distinguishable from other isoforms of the activatable element that are present in the sample.

In certain embodiments, multiple detectable analyte-specific binding agents are employed in a method in accordance with the present invention. By “multiple analyte-specific binding agents” is meant that at least 2 or more analyte-specific binding agents are used, including 3 or more, 4 or more, 5 or more, etc. In certain embodiments, each of the different analyte-specific binding agents are labeled (again, either directly or indirectly) with a distinctly detectable label (e.g., fluorophores that have emission wavelengths that can be detected in distinct channels on a flow cytometer, with or without compensation). The multiple analyte-specific binding agents can bind to the same analyte in or on a cell (e.g., two antibodies that bind to different epitopes on the same protein), to different analytes in or on the cell, or in any combination (e.g., two agents that bind the same analyte and a third that binds to a distinct analyte). The upper limit for the number of analyte specific binding agents will depend largely on the parameters of the assay and the detection capacity of the detecting system employed.

Following the combining or pooling, and any desired subsequent treatment, e.g., by contacting with labeling reagents for phenotypic markers (e.g., as described above), the methods may include flow cytometrically assaying the assay composition. By “flow cytometrically assaying” is meant performing a flow cytometric assay on a composition, e.g., an assay composition as described above. The flow cytometric assaying may include characterizing a sample, e.g., a sample including the assay composition, with a flow cytometer system. The flow cytometric assaying may include introducing the assay composition into a flow cytometer. A flow cytometer typically includes a sample reservoir for receiving a fluid sample, such as a sample including the assay composition, and a sheath reservoir containing a sheath fluid. The flow cytometer transports the particles (including cells, e.g., from the assay composition) in the fluid sample as a cell stream to a flow cell, while also directing the sheath fluid to the flow cell. To characterize the components of the flow stream, the flow stream is irradiated with light. Variations in the materials in the flow stream, such as morphologies or the presence of fluorescent labels, may cause variations in the observed light and these variations allow for characterization and separation. For example, particles, such as molecules, analyte-bound beads, or individual cells, in a fluid suspension are passed by a detection region in which the particles are exposed to an excitation light, typically from one or more lasers, and the light scattering and fluorescence properties of the particles are measured. Particles or components thereof typically are labeled with fluorescent dyes to facilitate detection. A multiplicity of different particles or components may be simultaneously detected by using spectrally distinct fluorescent dyes to label the different particles or components. In some implementations, a multiplicity of detectors, one for each of the scatter parameters to be measured, and one or more for each of the distinct dyes to be detected are included in the analyzer. For example, some embodiments include spectral configurations where more than one sensor or detector is used per dye. The data obtained include the signals measured for each of the light scatter detectors and the fluorescence emissions. In certain embodiments, the flow cytometric assay may detect a signal indicating the presence of the labeled secondary antibody in the sample. Where a signal is detected, the sample may include an antibody (antibodies) to the antigenic determinant of the coronaviral antigen.

As summarized above, a sample (e.g., in a flow stream of the flow cytometer) may be irradiated with light from a light source. In some embodiments, the light source is a broadband light source, emitting light having a broad range of wavelengths, such as for example, spanning 50 nm or more, such as 100 nm or more, such as 150 nm or more, such as 200 nm or more, such as 250 nm or more, such as 300 nm or more, such as 350 nm or more, such as 400 nm or more and including spanning 500 nm or more. For example, one suitable broadband light source emits light having wavelengths from 200 nm to 1500 nm. Another example of a suitable broadband light source includes a light source that emits light having wavelengths from 400 nm to 1000 nm. Where methods include irradiating with a broadband light source, broadband light source protocols of interest may include, but are not limited to, a halogen lamp, deuterium arc lamp, xenon arc lamp, stabilized fiber-coupled broadband light source, a broadband LED with continuous spectrum, superluminescent emitting diode, semiconductor light emitting diode, wide spectrum LED white light source, an multi-LED integrated white light source, among other broadband light sources or any combination thereof.

In other embodiments, methods includes irradiating with a narrow band light source emitting a particular wavelength or a narrow range of wavelengths, such as for example with a light source which emits light in a narrow range of wavelengths like a range of 50 nm or less, such as 40 nm or less, such as 30 nm or less, such as 25 nm or less, such as 20 nm or less, such as 15 nm or less, such as 10 nm or less, such as 5 nm or less, such as 2 nm or less and including light sources which emit a specific wavelength of light (i.e., monochromatic light). Where methods include irradiating with a narrow band light source, narrow band light source protocols of interest may include, but are not limited to, a narrow wavelength LED, laser diode or a broadband light source coupled to one or more optical bandpass filters, diffraction gratings, monochromators or any combination thereof.

In certain embodiments, methods include irradiating the sample with one or more lasers. As discussed above, the type and number of lasers will vary depending on the sample as well as desired light collected and may be a gas laser, such as a helium-neon laser, argon laser, krypton laser, xenon laser, nitrogen laser, CO₂ laser, CO laser, argon-fluorine (ArF) excimer laser, krypton-fluorine (KrF) excimer laser, xenon chlorine (XeCl) excimer laser or xenon-fluorine (XeF) excimer laser or a combination thereof. In others instances, the methods include irradiating the flow stream with a dye laser, such as a stilbene, coumarin or rhodamine laser. In yet other instances, methods include irradiating the flow stream with a metal-vapor laser, such as a helium-cadmium (HeCd) laser, helium-mercury (HeHg) laser, helium-selenium (HeSe) laser, helium-silver (HeAg) laser, strontium laser, neon-copper (NeCu) laser, copper laser or gold laser and combinations thereof. In still other instances, methods include irradiating the flow stream with a solid-state laser, such as a ruby laser, an Nd:YAG laser, NdCrYAG laser, Er:YAG laser, Nd:YLF laser, Nd:YVO₄ laser, Nd:YCa₄O(BO₃)₃ laser, Nd:YCOB laser, titanium sapphire laser, thulim YAG laser, ytterbium YAG laser, ytterbium₂O₃ laser or cerium doped lasers and combinations thereof.

The sample may be irradiated with one or more of the above mentioned light sources, such as 2 or more light sources, such as 3 or more light sources, such as 4 or more light sources, such as 5 or more light sources and including 10 or more light sources. The light source may include any combination of types of light sources. For example, in some embodiments, the methods include irradiating the sample in the flow stream with an array of lasers, such as an array having one or more gas lasers, one or more dye lasers and one or more solid-state lasers. Where desired, at least one laser will be used for excitation of the fluorescent barcodes, and other lasers for other fluorophores associated with the cells.

In certain instances, the flow stream is irradiated with a plurality of beams of frequency-shifted light and a cell in the flow stream is imaged by fluorescence imaging using radiofrequency tagged emission (FIRE) to generate a frequency-encoded image, such as those described in Diebold, et al. Nature Photonics Vol. 7(10); 806-810 (2013) as well as described in U.S. Pat. Nos. 9,423,353; 9,784,661 and 10,006,852 and U.S. Patent Publication Nos. 2017/0133857 and 2017/0350803, the disclosures of which are herein incorporated by reference.

Aspects of the present methods include collecting fluorescent light with a fluorescent light detector. A fluorescent light detector may, in some instances, be configured to detect fluorescence emissions from fluorescent molecules, e.g., labeled specific binding members (such as labeled antibodies that specifically bind to markers of interest) associated with the particle in the flow cell. In certain embodiments, methods include detecting fluorescence from the sample with one or more fluorescent light detectors, such as 2 or more, such as 3 or more, such as 4 or more, such as 5 or more, such as 6 or more, such as 7 or more, such as 8 or more, such as 9 or more, such as or more, such as 15 or more and including 25 or more fluorescent light detectors. In embodiments, each of the fluorescent light detectors is configured to generate a fluorescence data signal. Fluorescence from the sample may be detected by each fluorescent light detector, independently, over one or more of the wavelength ranges of 200 nm-1200 nm. In some instances, methods include detecting fluorescence from the sample over a range of wavelengths, such as from 200 nm to 1200 nm, such as from 300 nm to 1100 nm, such as from 400 nm to 1000 nm, such as from 500 nm to 900 nm and including from 600 nm to 800 nm. In other instances, methods include detecting fluorescence with each fluorescence detector at one or more specific wavelengths. For example, the fluorescence may be detected at one or more of 450 nm, 518 nm, 519 nm, 561 nm, 578 nm, 605 nm, 607 nm, 625 nm, 650 nm, 660 nm, 667 nm, 670 nm, 668 nm, 695 nm, 710 nm, 723 nm, 780 nm, 785 nm, 647 nm, 617 nm and any combinations thereof, depending on the number of different fluorescent light detectors in the subject light detection system. In certain embodiments, methods include detecting wavelengths of light which correspond to the fluorescence peak wavelength of certain fluorophores present in the sample. In embodiments, fluorescent flow cytometer data is received from one or more fluorescent light detectors (e.g., one or more detection channels), such as 2 or more, such as 3 or more, such as 4 or more, such as or more, such as 6 or more and including 8 or more fluorescent light detectors (e.g., 8 or more detection channels).

Light from the sample may be measured at one or more wavelengths of, such as at 5 or more different wavelengths, such as at 10 or more different wavelengths, such as at 25 or more different wavelengths, such as at 50 or more different wavelengths, such as at 100 or more different wavelengths, such as at 200 or more different wavelengths, such as at 300 or more different wavelengths and including measuring the collected light at 400 or more different wavelengths.

In certain embodiments, methods include spectrally resolving the light from each fluorophore of the fluorophore-biomolecule reagent pairs in the sample. In some embodiments, the overlap between each different fluorophore is determined and the contribution of each fluorophore to the overlapping fluorescence is calculated. In some embodiments, spectrally resolving light from each fluorophore includes calculating a spectral unmixing matrix for the fluorescence spectra for each of the plurality of fluorophores having overlapping fluorescence in the sample detected by the light detection system. In certain instances, spectrally resolving the light from each fluorophore and calculating a spectral unmixing matrix for each fluorophore may be used to estimate the abundance of each fluorophore, such as for example to resolve the abundance of target cells in the sample.

In certain embodiments, methods include spectrally resolving light detected by a plurality of photodetectors such as described e.g., in International Patent Application No. PCT/US2019/068395 filed on Dec. 23, 2019; U.S. Provisional Patent Application No. 62/971,840 filed on Feb. 7, 2020 and U.S. Provisional Patent Application No. 63/010,890 filed on Apr. 16, 2020, the disclosures of which are herein incorporated by reference in their entirety. For example, spectrally resolving light detected by the plurality of photodetectors of the second set of photodetectors may be include solving a spectral unmixing matrix using one or more of: 1) a weighted least square algorithm; 2) a Sherman-Morrison iterative inverse updater; 3) an LU matrix decomposition, such as where a matrix is decomposed into a product of a lower-triangular (L) matrix and an upper-triangular (U) matrix; 4) a modified Cholesky decomposition; 5) by OR factorization; and 6) calculating a weighted least squares algorithm by singular value decomposition.

In certain embodiments, methods further include characterizing the spillover spreading of the light detected by a plurality of photodetectors such as described e.g., in U.S. patent application Ser. No. 17/237,504, the disclosure of which is herein incorporated by reference.

In certain instances, the abundance of fluorophores associated with (e.g., chemically associated (i.e., covalently, ionically) or physically associated) a target particle is calculated from the spectrally resolved light from each fluorophore associated with the particle. For instance, in one example the relative abundance of each fluorophore associated with a target particle is calculated from the spectrally resolved light from each fluorophore. In another example, the absolute abundance of each fluorophore associated with the target particle is calculated from the spectrally resolved light from each fluorophore. In certain embodiments, a particle may be identified or classified based on the relative abundance of each fluorophore determined to be associated with the particle. In these embodiments, the particle may be identified or classified by any convenient protocol such as by: comparing the relative or absolute abundance of each fluorophore associated with a particle with a control sample having particles of known identity; or by conducting spectroscopic or other assay analysis of a population of particles (e.g., cells) having the calculated relative or absolute abundance of associated fluorophores.

In certain embodiments, methods include sorting one or more of the particles (e.g., cells) of the sample that are identified based on the estimated abundance of the fluorophores associated with the particle. The term “sorting” is used herein in its conventional sense to refer to separating components (e.g., droplets containing cells, droplets containing non-cellular particles such as biological macromolecules) of a sample and in some instances, delivering the separated components to one or more sample collection containers. For example, methods may include sorting 2 or more components of the sample, such as 3 or more components, such as 4 or more components, such as 5 or more components, such as 10 or more components, such as 15 or more components and including sorting 25 or more components of the sample. In sorting particles identified based on the abundance of fluorophores associated with the particle, methods include data acquisition, analysis and recording, such as with a computer, where multiple data channels record data from each detector used in obtaining the overlapping spectra of the plurality of fluorophore-biomolecule reagent pairs associated with the particle. In these embodiments, analysis includes spectrally resolving light (e.g., by calculating the spectral unmixing matrix) from the plurality of fluorophores of the fluorophore-biomolecule reagent pairs having overlapping spectra that are associated with the particle and identifying the particle based on the estimated abundance of each fluorophore associated with the particle. This analysis may be conveyed to a sorting system which is configured to generate a set of digitized parameters based on the particle classification. In some embodiments, methods for sorting components of a sample include sorting particles (e.g., cells in a biological sample), such as described in U.S. Pat. Nos. 3,960,449; 4,347,935; 4,667,830; 5,245,318; 5,464,581; 5,483,469; 5,602,039; 5,643,796; 5,700,692; 6,372,506 and 6,809,804, the disclosures of which are herein incorporated by reference. In some embodiments, methods include sorting components of the sample with a particle sorting module, such as those described in U.S. Pat. Nos. 9,551,643 and 10,324,019, U.S. Patent Publication No. 2017/0299493 and International Patent Publication No. WO/2017/040151, the disclosure of which is incorporated herein by reference. In certain embodiments, cells of the sample are sorted using a sort decision module having a plurality of sort decision units, such as those described in U.S. patent application Ser. No. 16/725,756, filed on Dec. 23, 2019, the disclosure of which is incorporated herein by reference.

Flow cytometric assay procedures are well known in the art. See, e.g., Ormerod (ed.), Flow Cytometry: A Practical Approach, Oxford Univ. Press (1997); Jaroszeski et al. (eds.), Flow Cytometry Protocols, Methods in Molecular Biology No. 91, Humana Press (1997); Practical Flow Cytometry, 3rd ed., Wiley-Liss (1995); Virgo, et al. (2012) Ann Clin Biochem. January; 49(pt 1):17-28; Linden, et. al., Semin Throm Hemost. 2004 October; 30(5):502-11; Alison, et al. J Pathol, 2010 December; 222(4):335-344; and Herbig, et al. (2007) Crit Rev Ther Drug Carrier Syst. 24(3):203-255; the disclosures of which are incorporated herein by reference. In certain aspects, flow cytometrically assaying the composition involves using a flow cytometer capable of simultaneous excitation and detection of multiple fluorophores, such as a BD Biosciences FACSCanto™ flow cytometer, used substantially according to the manufacturer's instructions. Methods of the present disclosure may involve image cytometry, such as is described in Holden et al. (2005) Nature Methods 2:773 and Valet, et al. 2004 Cytometry 59:167-171, the disclosures of which are incorporated herein by reference.

As discussed above, the method includes cytometric analysis which may include sorting. Cells of interest identified in the sample may be sorted and subsequently analyzed by any convenient analysis technique. Subsequent analysis techniques of interest include, but are not limited to, sequencing; assaying by CellSearch, as described in Food and Drug Administration (2004) Final rule. Fed Regist 69: 26036-26038; assaying by CTC Chip, as described in Nagrath, et al. (2007) Nature 450: 1235-1239; assaying by MagSweeper, as described in Talasaz, et al. (2009). Proc Natl Acad Sci USA 106: 3970-3975; and assaying by nanostructured substrates, as described in Wang S, et al. (2011) Angew Chem Int Ed Engl 50: 3084-3088; the disclosures of which are incorporated herein by reference. Where desired, the sorting protocol may include distinguishing viable and dead cells, where any convenient staining protocol for identifying such cells may be incorporated into the methods.

Analysis of the data acquired from a barcoded sample of the invention may include deconvolution. By “deconvolution” is meant a process, whether performed manually or in an automated system, by which the detected fluorescent barcode of each cell is used to determine from which original sample it was derived. Because the type and amount of each fluorescent barcode for each of the starting samples is known, the detected fluorescent barcode signature of each cell (i.e., its barcode signature) can be used to positively identify its sample of origin. Deconvolution of multiplexed data can be done using any convenient method, including using computer-based analysis software known in the art (e.g., FlowJo software package, available from BD Biosciences). Deconvolution can be done manually (e.g., viewing the data and categorizing the cells by hand), automatically (e.g., by employing data analysis software configured specifically to deconvolute barcoded data), or a combination thereof. In certain embodiments, computer programs can be employed to create individual data files for each of the deconvoluted barcoded samples which correspond to the original starting samples for ease of data manipulation and/or interpretation.

Analysis of the data acquired from a barcoded multiplexed sample of the invention involves analyzing the cells for the detectable characteristic(s) of interest (e.g., as described in greater detail above). Analysis of the detectable characteristic may be done at any convenient step in the data analysis phase, including before, during or after deconvolution. Indeed, because the acquired data can be analyzed and re-analyzed at will, no limitation with regard to the order of deconvolution and analysis of the detectable characteristic(s) is intended.

### Kits

Aspects of the invention further include kits and compositions that find use in practicing various embodiments of methods of the invention. Kits of the invention may include a plurality of distinguishably fluorescently labeled specific binding members that specifically bind to universal cell surface markers, e.g., as described above. The different distinguishably fluorescently labeled specific binding members may be separate or present as precombined labeling compositions, as desired. The kits may further include one or more additional components finding use in practicing embodiments of the methods. For example, the kits may include beads of a multiplex bead array assay, e.g., as described above, where such beads may include specific binding members for analytes of interest and one or more surface markers, e.g., as described above. Kits may also include components employed various workflows, e.g., multi-well plates, liquid containers, e.g., tubes, etc. Furthermore, the kits may include one or more reagents employed in flow cytometric workflow, e.g., labeling reagents, buffers, dyes, etc. Components of the kits may be present in separate containers, or multiple components may be present in a single container.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), portable flash drive, and the like, on which the information has been recorded. Yet another form of these instructions that may be present is a website address which may be used via the internet to access the information at a removed site.

The following is offered by way of illustration and not by way of limitation.

### EXPERIMENTAL

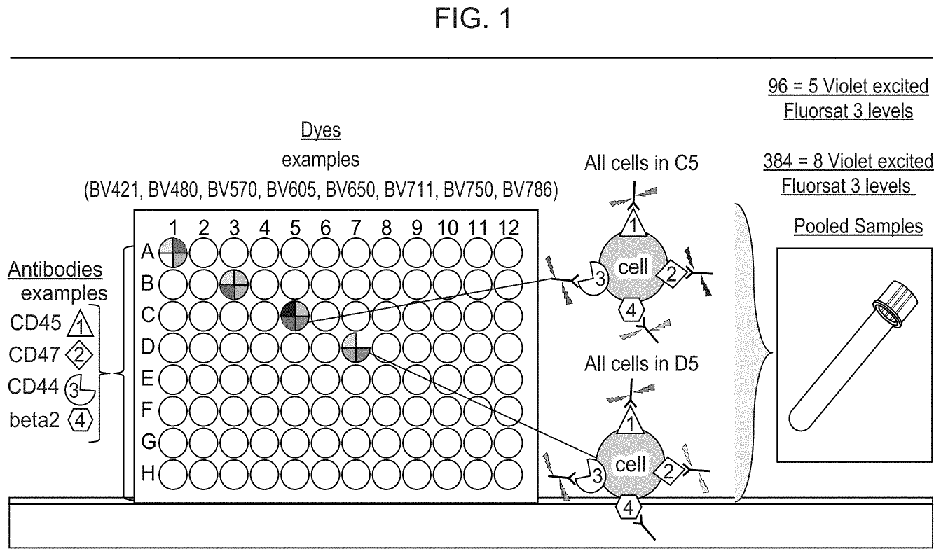

An embodiment of a workflow according to the invention is illustrated in FIG. 1. As shown, cellular samples in different wells of a multi-well plate are barcoded with different combinations of fluorescently labeled antibodies that specifically bind to different universal cell surface markers. The different fluorophores are all violet excitable.

Notwithstanding the appended claims, the disclosure is also defined by the following clauses:
1. A method of producing a plurality of distinguishably fluorescently barcoded cellular samples, the method comprising:
   providing a plurality of cellular samples; and
   labeling different cellular samples of the plurality with unique fluorescent barcodes, wherein a given fluorescent barcode comprises one or more fluorescently labeled specific binding members that specifically bind to a universal cell marker; to produce a plurality of distinguishably fluorescently barcoded cellular samples.
2. The method according to Clause 1, wherein the plurality of cellular samples comprises 5 to 500 cellular samples.
3. The method according to Clause 2, wherein the plurality of cellular samples comprises 50 to 400 cellular samples.
4. The method according to Clause 3, wherein the plurality of cellular samples comprises 96 cellular samples.
5. The method according to Clause 3, wherein the plurality of cellular samples comprises 384 cellular samples.
6. The method according to any of the preceding clauses, wherein the cellular samples are provided in wells of a multi-well plate.
7. The method according to any of the preceding clauses, wherein each unique fluorescent barcode comprises a plurality of distinguishably fluorescently labeled specific binding members.
8. The method according to Clause 7, wherein the plurality of distinguishably fluorescently labeled specific binding members comprises 2 to 20 distinguishably fluorescently labeled specific binding members.
9. The method according to Clause 8, wherein the plurality of distinguishably fluorescently labeled specific binding members comprises 3 to 10 distinguishably fluorescently labeled specific binding members.
10. The method according to any of Clauses 7 to 9, wherein each of the plurality of distinguishably fluorescently labeled specific binding members differs from each other by one or more of emission maximum and brightness.
11. The method according to any of Clauses 7 to 10, wherein each of the plurality of distinguishably fluorescently labeled specific binding members is excitable by common light source.
12. The method according to Clause 11, wherein the common light source is a laser.
13. The method according to any of the preceding clauses, wherein the universal cell marker is a non-phenotype marker.
14. The method according to Clause 13, wherein the universal cell marker is selected from the group consisting of: CD44, CD45, CD47 and β-2 micro-globulin.
15. The method according to any of the preceding clauses, wherein the specific binding member is an antibody or binding fragment thereof.
16. The method according to any of the preceding clauses, wherein each cellular sample comprises from 50 to 50,000,000 cells.
17. The method according to any of the preceding clauses, wherein the method further comprises pooling the plurality of distinguishably fluorescently labeled barcoded samples to produce a pooled sample.
18. The method according to Clause 16, wherein the method further comprises flow cytometrically assaying the pooled sample.
19. The method according to Clause 18, wherein the method further comprises assigning cells having the same fluorescent barcode as originating from the same cellular sample.
20. The method according to any of the preceding clauses, wherein the method further comprises differentially treating two or more of the plurality of cellular samples.
21. A plurality of distinguishably fluorescently barcoded cellular samples each labeled with a unique fluorescent barcode, wherein a given fluorescent barcode comprises one or more fluorescently labeled specific binding members that specifically bind to a universal cell marker
22. The plurality of distinguishably fluorescently barcoded cellular samples according to Clause 21, wherein the plurality comprises 5 to 500 distinguishably fluorescently barcoded samples cellular samples.
23. The plurality of distinguishably fluorescently barcoded cellular samples according to Clause 22, wherein the plurality comprises 50 to 400 distinguishably fluorescently barcoded cellular samples.
24. The plurality of distinguishably fluorescently barcoded cellular samples according to Clause 23, wherein the plurality comprises 96 distinguishably fluorescently barcoded cellular samples.
25. The plurality of distinguishably fluorescently barcoded cellular samples according to Clause 23, wherein the plurality comprises 384 distinguishably fluorescently barcoded cellular samples.
26. The plurality of distinguishably fluorescently barcoded cellular samples according to any of Clauses 21 to 25, wherein the cellular samples are provided in wells of a multi-well plate.
27. The plurality of distinguishably fluorescently barcoded cellular samples according to any of Clauses 21 to 26, wherein each unique fluorescent barcode comprises a plurality of distinguishably fluorescently labeled specific binding members.
28. The plurality of distinguishably fluorescently barcoded cellular samples according to Clause 27, wherein the plurality of distinguishably fluorescently labeled specific binding members comprises 2 to 20 distinguishably fluorescently labeled specific binding members.
29. The plurality of distinguishably fluorescently barcoded cellular samples according to Clause 28, wherein the plurality of distinguishably fluorescently labeled specific binding members comprises 5 to 10 distinguishably fluorescently labeled specific binding members.
30. The plurality of distinguishably fluorescently barcoded cellular samples according to any of Clauses 27 to 29, wherein each of the plurality of distinguishably fluorescently labeled specific binding members differs from each other by one or more of emission maximum and brightness.
31. The plurality of distinguishably fluorescently barcoded cellular samples according to any of Clauses 27 to 30, wherein each of the plurality of distinguishably fluorescently labeled specific binding members is excitable by common light source.
32. The plurality of distinguishably fluorescently barcoded cellular samples according to Clause 31, wherein the common light source is a laser.
33. The plurality of distinguishably fluorescently barcoded cellular samples according to any of Clauses 21 to 32, wherein the universal cell marker is a non-phenotype marker.
34. The plurality of distinguishably fluorescently barcoded cellular samples according to Clause 33, wherein the universal cell marker is selected from the group consisting of: CD44, CD45, CD47 and β-2 micro-globulin.
35. The plurality of distinguishably fluorescently barcoded cellular samples according to any of Clauses 21 to 34, wherein the specific binding member is an antibody or binding fragment thereof.
36. The plurality of distinguishably fluorescently barcoded cellular samples according to any of Clauses 21 to 35, wherein each cellular sample comprises from 50 to 50,000,000 cells.
37. A pooled sample comprising a plurality of distinguishably fluorescently barcoded cellular samples according to any of Clauses 21 to 36.
38. A flow cytometer comprising a pooled sample according to Clause 37.
39. A kit comprising:
   a plurality of distinguishably fluorescently labeled specific binding members that specifically bind to universal cell surface markers.
40. The kit according to Clause 39, wherein the plurality comprises 2 to 20 distinguishably fluorescently labeled specific binding members.
41. The kit according to Clause 40, wherein the plurality comprises 3 to 10 distinguishably fluorescently labeled specific binding members.
42. The kit according to any of Clauses 39 to 41, wherein each of the plurality of distinguishably fluorescently labeled specific binding members differs from each other by one or more of emission maximum and brightness.
43. The kit according to any of Clauses 39 to 42, wherein each of the plurality of distinguishably fluorescently labeled specific binding members is excitable by common light source.
44. The kit according to Clause 43, wherein the common light source is a laser.
45. The kit according to any of Clauses 39 to 44, wherein the universal cell marker is a non-phenotype marker.
46. The kit according to Clause 45, wherein the universal cell marker is selected from the group consisting of: CD44, CD45, CD47 and β-2 micro-globulin.
47. The kit according to any of Clauses 39 to 46, wherein the plurality of distinguishably fluorescently labeled specific binding members comprises distinguishably fluorescently labeled specific binding members that specifically bind to different universal cell surface markers.
48. The kit according to any of Clauses 39 to 47, wherein the specific binding member is an antibody or binding fragment thereof.

Notwithstanding the appended claims, the disclosure is also defined by the following clauses:
1. A method of producing a plurality of distinguishably fluorescently barcoded particle samples, the method comprising:
   providing a plurality of particle samples; and
   labeling different particle samples of the plurality with unique fluorescent barcodes, wherein a given fluorescent barcode comprises one or more fluorescently labeled specific binding members that specifically bind to a particle marker;
   to produce a plurality of distinguishably fluorescently barcoded particle samples.
2. The method according to Clause 1, wherein the plurality of particle samples comprises 5 to 500 particle samples.
3. The method according to Clause 2, wherein the plurality of particle samples comprises 50 to 400 particle samples.
4. The method according to Clause 3, wherein the plurality of particle samples comprises 96 particle samples.
5. The method according to Clause 3, wherein the plurality of particle samples comprises 384 particle samples.
6. The method according to any of the preceding clauses, wherein the particle samples are provided in wells of a multi-well plate.
7. The method according to any of the preceding clauses, wherein each unique fluorescent barcode comprises a plurality of distinguishably fluorescently labeled specific binding members.
8. The method according to Clause 7, wherein the plurality of distinguishably fluorescently labeled specific binding members comprises 2 to 20 distinguishably fluorescently labeled specific binding members.
9. The method according to Clause 8, wherein the plurality of distinguishably fluorescently labeled specific binding members comprises 3 to 10 distinguishably fluorescently labeled specific binding members.
10. The method according to any of Clauses 7 to 9, wherein each of the plurality of distinguishably fluorescently labeled specific binding members differs from each other by one or more of emission maximum and brightness.
11. The method according to any of Clauses 7 to 10, wherein each of the plurality of distinguishably fluorescently labeled specific binding members is excitable by common light source.
12. The method according to Clause 11, wherein the common light source is a laser.
13. The method according to any of the preceding clauses, wherein the marker is a non-phenotype marker.
14. The method according to Clause 13, wherein the marker is selected from the group consisting of: CD44, CD45, CD47 and β-2 micro-globulin.
15. The method according to any of the preceding clauses, wherein the specific binding member is an antibody or binding fragment thereof.
16. The method according to any of the preceding clauses, wherein each particle sample comprises from 50 to 50,000,000 particles.
17. The method according to any of the preceding clauses, wherein the method further comprises pooling the plurality of distinguishably fluorescently labeled barcoded samples to produce a pooled sample.
18. The method according to Clause 16, wherein the method further comprises flow cytometrically assaying the pooled sample.
19. The method according to Clause 18, wherein the method further comprises assigning particles having the same fluorescent barcode as originating from the same particle sample.
20. The method according to any of the preceding clauses, wherein the method further comprises differentially treating two or more of the plurality of particle samples.
21. A plurality of distinguishably fluorescently barcoded particles samples each labeled with a unique fluorescent barcode, wherein a given fluorescent barcode comprises one or more fluorescently labeled specific binding members that specifically bind to a particle marker
22. The plurality of distinguishably fluorescently barcoded particle samples according to Clause 21, wherein the plurality comprises 5 to 500 distinguishably fluorescently barcoded particle samples.
23. The plurality of distinguishably fluorescently barcoded particle samples according to Clause 22, wherein the plurality comprises 50 to 400 distinguishably fluorescently barcoded particle samples.
24. The plurality of distinguishably fluorescently barcoded particle samples according to Clause 23, wherein the plurality comprises 96 distinguishably fluorescently barcoded particle samples.
25. The plurality of distinguishably fluorescently barcoded particle samples according to Clause 23, wherein the plurality comprises 384 distinguishably fluorescently barcoded particle samples.
26. The plurality of distinguishably fluorescently barcoded particle samples according to any of Clauses 21 to 25, wherein the particle samples are provided in wells of a multi-well plate.
27. The plurality of distinguishably fluorescently barcoded particle samples according to any of Clauses 21 to 26, wherein each unique fluorescent barcode comprises a plurality of distinguishably fluorescently labeled specific binding members.
28. The plurality of distinguishably fluorescently barcoded particle samples according to Clause 27, wherein the plurality of distinguishably fluorescently labeled specific binding members comprises 2 to 20 distinguishably fluorescently labeled specific binding members.
29. The plurality of distinguishably fluorescently barcoded particle samples according to Clause 28, wherein the plurality of distinguishably fluorescently labeled specific binding members comprises 5 to 10 distinguishably fluorescently labeled specific binding members.
30. The plurality of distinguishably fluorescently barcoded particle samples according to any of Clauses 27 to 29, wherein each of the plurality of distinguishably fluorescently labeled specific binding members differs from each other by one or more of emission maximum and brightness.
31. The plurality of distinguishably fluorescently barcoded particle samples according to any of Clauses 27 to 30, wherein each of the plurality of distinguishably fluorescently labeled specific binding members is excitable by common light source.
32. The plurality of distinguishably fluorescently barcoded particle samples according to Clause 31, wherein the common light source is a laser.
33. The plurality of distinguishably fluorescently barcoded particle samples according to any of Clauses 21 to 32, wherein the particle marker is a non-phenotype marker.
34. The plurality of distinguishably fluorescently barcoded particle samples according to Clause 33, wherein the particle marker is selected from the group consisting of: CD44, CD45, CD47 and β-2 micro-globulin.
35. The plurality of distinguishably fluorescently barcoded particle samples according to any of Clauses 21 to 34, wherein the specific binding member is an antibody or binding fragment thereof.
36. The plurality of distinguishably fluorescently barcoded particle samples according to any of Clauses 21 to 35, wherein each cellular sample comprises from 50 to 50,000,000 cells.
37. A pooled sample comprising a plurality of distinguishably fluorescently barcoded particle samples according to any of Clauses 21 to 36.
38. A flow cytometer comprising a pooled sample according to Clause 37.
39. A kit comprising:
   a plurality of distinguishably fluorescently labeled specific binding members that specifically bind to particle markers.
40. The kit according to Clause 39, wherein the plurality comprises 2 to 20 distinguishably fluorescently labeled specific binding members.
41. The kit according to Clause 40, wherein the plurality comprises 3 to 10 distinguishably fluorescently labeled specific binding members.
42. The kit according to any of Clauses 39 to 41, wherein each of the plurality of distinguishably fluorescently labeled specific binding members differs from each other by one or more of emission maximum and brightness.
43. The kit according to any of Clauses 39 to 42, wherein each of the plurality of distinguishably fluorescently labeled specific binding members is excitable by common light source.
44. The kit according to Clause 43, wherein the common light source is a laser.
45. The kit according to any of Clauses 39 to 44, wherein the particle marker is a non-phenotype marker.
46. The kit according to Clause 45, wherein the particle marker is selected from the group consisting of: CD44, CD45, CD47 and β-2 micro-globulin.
47. The kit according to any of Clauses 39 to 46, wherein the plurality of distinguishably fluorescently labeled specific binding members comprises distinguishably fluorescently labeled specific binding members that specifically bind to different particle markers.
48. The kit according to any of Clauses 39 to 47, wherein the specific binding member is an antibody or binding fragment thereof.
49. The kit according to any of Clauses 39 to 47, wherein the kit further comprises beads of a multiplex bead array assay.

Notwithstanding the appended claims, the disclosure is also defined by the following clauses:
1. A method of producing a plurality of distinguishably fluorescently barcoded bead samples, the method comprising:
   providing a plurality of bead samples; and
   labeling different particle samples of the plurality with unique fluorescent barcodes, wherein a given fluorescent barcode comprises one or more fluorescently labeled specific binding members that specifically bind to a bead marker;
   to produce a plurality of distinguishably fluorescently barcoded bead samples.
2. The method according to Clause 1, wherein the plurality of bead samples comprises 5 to 500 bead samples.
3. The method according to Clause 2, wherein the plurality of bead samples comprises 50 to 400 bead samples.
4. The method according to Clause 3, wherein the plurality of bead samples comprises 96 bead samples.
5. The method according to Clause 3, wherein the plurality of bead samples comprises 384 bead samples.
6. The method according to any of the preceding clauses, wherein the bead samples are provided in wells of a multi-well plate.
7. The method according to any of the preceding clauses, wherein each unique fluorescent barcode comprises a plurality of distinguishably fluorescently labeled specific binding members.
8. The method according to Clause 7, wherein the plurality of distinguishably fluorescently labeled specific binding members comprises 2 to 20 distinguishably fluorescently labeled specific binding members.
9. The method according to Clause 8, wherein the plurality of distinguishably fluorescently labeled specific binding members comprises 3 to 10 distinguishably fluorescently labeled specific binding members.
10. The method according to any of Clauses 7 to 9, wherein each of the plurality of distinguishably fluorescently labeled specific binding members differs from each other by one or more of emission maximum and brightness.
11. The method according to any of Clauses 7 to 10, wherein each of the plurality of distinguishably fluorescently labeled specific binding members is excitable by common light source.
12. The method according to Clause 11, wherein the common light source is a laser.
13. The method according to any of the preceding clauses, wherein the marker is a non-phenotype marker.
14. The method according to Clause 13, wherein the marker is selected from the group consisting of: CD44, CD45, CD47 and β-2 micro-globulin.
15. The method according to any of the preceding clauses, wherein the specific binding member is an antibody or binding fragment thereof.
16. The method according to any of the preceding clauses, wherein each bead sample comprises from 50 to 50,000,000 bead.
17. The method according to any of the preceding clauses, wherein the method further comprises pooling the plurality of distinguishably fluorescently labeled barcoded samples to produce a pooled sample.
18. The method according to Clause 16, wherein the method further comprises flow cytometrically assaying the pooled sample.
19. The method according to Clause 18, wherein the method further comprises assigning bead having the same fluorescent barcode as originating from the same bead sample.
20. The method according to any of the preceding clauses, wherein the method further comprises differentially treating two or more of the plurality of bead samples.
21. A plurality of distinguishably fluorescently barcoded bead samples each labeled with a unique fluorescent barcode, wherein a given fluorescent barcode comprises one or more fluorescently labeled specific binding members that specifically bind to a bead marker.
22. The plurality of distinguishably fluorescently barcoded bead samples according to Clause 21, wherein the plurality comprises 5 to 500 distinguishably fluorescently barcoded bead samples.
23. The plurality of distinguishably fluorescently barcoded bead samples according to Clause 22, wherein the plurality comprises 50 to 400 distinguishably fluorescently barcoded bead samples.
24. The plurality of distinguishably fluorescently barcoded bead samples according to Clause 23, wherein the plurality comprises 96 distinguishably fluorescently barcoded bead samples.
25. The plurality of distinguishably fluorescently barcoded bead samples according to Clause 23, wherein the plurality comprises 384 distinguishably fluorescently barcoded bead samples.
26. The plurality of distinguishably fluorescently barcoded bead samples according to any of Clauses 21 to 25, wherein the bead samples are provided in wells of a multi-well plate.
27. The plurality of distinguishably fluorescently barcoded bead samples according to any of Clauses 21 to 26, wherein each unique fluorescent barcode comprises a plurality of distinguishably fluorescently labeled specific binding members.
28. The plurality of distinguishably fluorescently barcoded bead samples according to Clause 27, wherein the plurality of distinguishably fluorescently labeled specific binding members comprises 2 to 20 distinguishably fluorescently labeled specific binding members.
29. The plurality of distinguishably fluorescently barcoded bead samples according to Clause 28, wherein the plurality of distinguishably fluorescently labeled specific binding members comprises 5 to 10 distinguishably fluorescently labeled specific binding members.
30. The plurality of distinguishably fluorescently barcoded bead samples according to any of Clauses 27 to 29, wherein each of the plurality of distinguishably fluorescently labeled specific binding members differs from each other by one or more of emission maximum and brightness.
31. The plurality of distinguishably fluorescently barcoded bead samples according to any of Clauses 27 to 30, wherein each of the plurality of distinguishably fluorescently labeled specific binding members is excitable by common light source.
32. The plurality of distinguishably fluorescently barcoded bead samples according to Clause 31, wherein the common light source is a laser.
33. The plurality of distinguishably fluorescently barcoded bead samples according to any of Clauses 21 to 32, wherein the particle marker is a non-phenotype marker.
34. The plurality of distinguishably fluorescently barcoded bead samples according to Clause 33, wherein the particle marker is selected from the group consisting of: CD44, CD45, CD47 and β-2 micro-globulin.
35. The plurality of distinguishably fluorescently barcoded bead samples according to any of Clauses 21 to 34, wherein the specific binding member is an antibody or binding fragment thereof.
36. The plurality of distinguishably fluorescently barcoded bead samples according to any of Clauses 21 to 35, wherein each bead sample comprises from 50 to 50,000,000 bead.
37. A pooled sample comprising a plurality of distinguishably fluorescently barcoded bead samples according to any of Clauses 21 to 36.
38. A flow cytometer comprising a pooled sample according to Clause 37.
39. A kit comprising:
   a plurality of distinguishably fluorescently labeled specific binding members that specifically bind to particle markers.
40. The kit according to Clause 39, wherein the plurality comprises 2 to 20 distinguishably fluorescently labeled specific binding members.
41. The kit according to Clause 40, wherein the plurality comprises 3 to 10 distinguishably fluorescently labeled specific binding members.
42. The kit according to any of Clauses 39 to 41, wherein each of the plurality of distinguishably fluorescently labeled specific binding members differs from each other by one or more of emission maximum and brightness.
43. The kit according to any of Clauses 39 to 42, wherein each of the plurality of distinguishably fluorescently labeled specific binding members is excitable by common light source.
44. The kit according to Clause 43, wherein the common light source is a laser.
45. The kit according to any of Clauses 39 to 44, wherein the particle marker is a non-phenotype marker.
46. The kit according to Clause 45, wherein the particle marker is selected from the group consisting of: CD44, CD45, CD47 and β-2 micro-globulin.
47. The kit according to any of Clauses 39 to 46, wherein the plurality of distinguishably fluorescently labeled specific binding members comprises distinguishably fluorescently labeled specific binding members that specifically bind to different particle markers.
48. The kit according to any of Clauses 39 to 47, wherein the specific binding member is an antibody or binding fragment thereof.
49. The kit according to any of Clauses 39 to 47, wherein the kit further comprises beads of a multiplex bead array assay.

Although the foregoing invention has been described in some detail by way of illustration and example for purposes of clarity of understanding, it is readily apparent to those of ordinary skill in the art in light of the teachings of this invention that some changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A method of producing a plurality of distinguishably fluorescently barcoded particle samples, the method comprising:
providing a plurality of particle samples; and
labeling different particle samples of the plurality with unique fluorescent barcodes, wherein a given fluorescent barcode comprises one or more fluorescently labeled specific binding members that specifically bind to a particle marker;
to produce a plurality of distinguishably fluorescently barcoded particle samples.

2. The method according to claim 1, wherein the plurality of particle samples comprises 5 to 500 particle samples.

3. The method according to claim 2, wherein the plurality of particle samples comprises 50 to 400 particle samples.

4. The method according to claim 1, wherein the plurality of particles samples comprises a plurality of cellular samples.

5. The method according to claim 1, wherein the plurality of particle samples comprises a plurality of bead samples.

6. The method according to claim 1, wherein the particles samples are provided in wells of a multi-well plate.

7. The method according to claim 1, wherein each unique fluorescent barcode comprises a plurality of distinguishably fluorescently labeled specific binding members.

8. The method according to claim 7, wherein the plurality of distinguishably fluorescently labeled specific binding members comprises 2 to 20 distinguishably fluorescently labeled specific binding members.

9. The method according to claim 8, wherein the plurality of distinguishably fluorescently labeled specific binding members comprises 3 to 10 distinguishably fluorescently labeled specific binding members.

10. The method according to claim 7, wherein each of the plurality of distinguishably fluorescently labeled specific binding members differs from each other by one or more of emission maximum and brightness.

11. The method according to claim 7, wherein each of the plurality of distinguishably fluorescently labeled specific binding members is excitable by common light source.

12. The method according to claim 11, wherein the common light source is a laser.

13. The method according to claim 1, wherein the particle marker is a non-phenotype marker.

14. The method according to claim 13, wherein the particle marker is selected from the group consisting of: CD44, CD45, CD47 and β-2 micro-globulin.

15. The method according to claim 1, wherein the specific binding member is an antibody or binding fragment thereof.

16. The method according to claim 1, wherein each particle sample comprises from 50 to 50,000,000 particles.

17. The method according to claim 1, wherein the method further comprises pooling the plurality of distinguishably fluorescently labeled barcoded samples to produce a pooled sample.

18. The method according to claim 16, wherein the method further comprises flow cytometrically assaying the pooled sample.

19. The method according to claim 18, wherein the method further comprises assigning cells having the same fluorescent barcode as originating from the same particle sample.

20. The method according to claim 1, wherein the method further comprises differentially treating two or more of the plurality of particle samples.

21-49. (canceled)
